(19) United States
(12) Patent Application Publication
(10) Pub. No.: US 2026/0157628 A1
(43) Pub. Date: Jun. 11, 2026

(54) ARTIFICIAL INTELLIGENCE FOR DIAGNOSIS OF OCULAR SYSTEMS

(71) Applicant: Ajman University, AJman (AE)

(72) Inventors: Houssam Mouhib Hammoud, Ajman (AE); Mohammad Yaman Basel Al Aref, Ajman (AE); Mayar Mohammad Al Jabouli, Ajman (AE); Mohamad Mouhib Hammoud, Ajman (AE); Wessam Ghassan Shehieb, Ajman (AE); Kamran Arshad, Ajman (AE); Buthyna Bader Edin Alshekha, Ajman (AE)

(73) Assignee: Ajman University, Ajman (AE)

(21) Appl. No.: 19/410,505

(22) Filed: Dec. 5, 2025

Related U.S. Application Data
(60) Provisional application No. 63/729,274, filed on Dec. 6, 2024.

(51) Int. Cl. A61B 3/14; A61B 3/12

(52) U.S. Cl. CPC A61B 3/14; A61B 3/12

(57) ABSTRACT

A method, includes receiving, by an apparatus, electronic information about an eye. The method includes cropping, by the apparatus, the electronic information. The method includes analyzing, by the apparatus, the electronic information. The method includes determining, by the computing system, that the electronic information is associated with one of: a healthy eye, or an unhealthy eye.

### BACKGROUND

Traditional diagnostic methods such as Fundus cameras and slit-lamps are used for diagnosing various ocular diseases. However, such systems are often limited by their cost and accessibility which hinders timely intervention especially in rural areas and indigent societies with limited resources.

### BRIEF DESCRIPTION OF DRAWINGS

FIGS. 1A, 1B, and 1C are diagrams of an example system;

FIG. 2 is a diagram of an example flow process;

FIG. 3 is a diagram of an example table;

FIG. 4 is a diagram of an example flow process;

FIG. 5 is a diagram of an example flow process;

FIG. 6 is a diagram of an example computer architecture;

FIG. 7 is a diagram of an example computer architecture;

FIG. 8 is a diagram of an example screenshot;

FIG. 9 is a diagram of an example graph;

FIG. 10 is a diagram of an example graph;

FIG. 11 is a diagram of an example table;

FIG. 12 is a diagram of example retinal imagery;

FIG. 13 is a diagram of an example computing networking system;

FIG. 14 is a diagram of an example computer; and

FIGS. 15A, 15B, and 15C are diagrams of an example holding device.

### DETAILED DESCRIPTION OF PREFERRED EMBODIMENTS

The following detailed description refers to the accompanying drawings. The same reference numbers in different drawings may identify the same or similar elements.

Systems, devices, and/or methods described herein are utilizes a mobile-based (e.g., a smart phone), cloud-based deep learning model (the ocular analysis system) and a three-dimensional printed multi-lens holder for fundus imaging designed for capturing live fundus images aimed at diagnostic classification. In embodiments, the acquired images undergo pre-processing before being used in a pre-trained deep learning model, which performs a detailed analysis to identify the presence of specific ocular diseases.

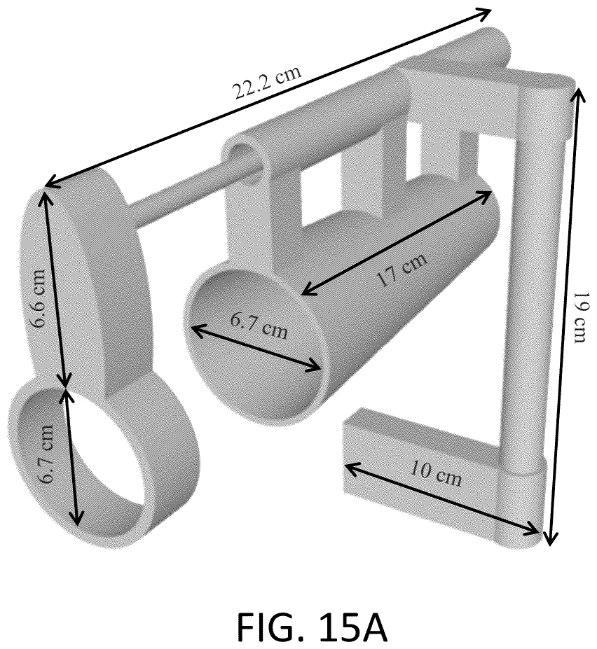

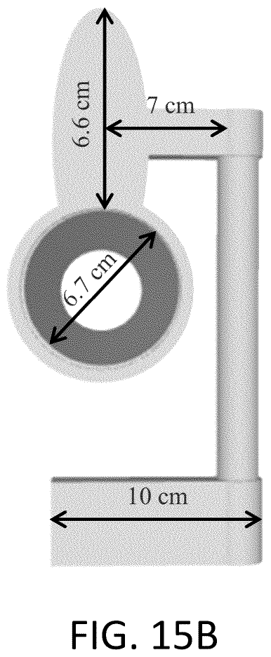

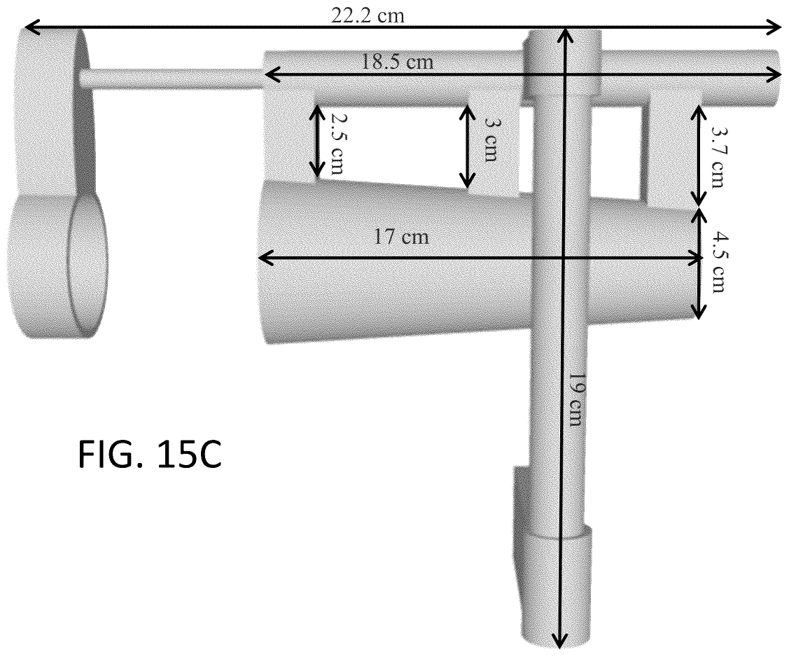

In embodiments, a three-dimensional (3D) printed apparatus is proposed which uses the smartphone as the camera alongside holders for multi-lenses as shown in FIG. 15. In embodiments, the apparatus allows for small positional adjustments for the smartphone, back and forth movement of the lenses and focuses the flashlight on the lenses to provide an optimal viewing of the retina of the eye.

In embodiments, the apparatus can accommodate multiple lens holders, a detachable tube for flashlight focusing and a smartphone holder. In embodiments, the apparatus can include metallic bearings were used to provide a convenient experience to the end-user while holding the smartphone device and to enable smooth movements of the lens holders.

In embodiments, the processes described herein may include five stages. In embodiments, the five stages include data acquisition; preprocessing and augmentation, training and testing (which includes cross-validation techniques in conjunction with a pre-trained ResNet Model, ensuring robust model development); post-training, rigorous model evaluation and assessing performance metrics to ascertain efficacy; and providing the result in an electronic application.

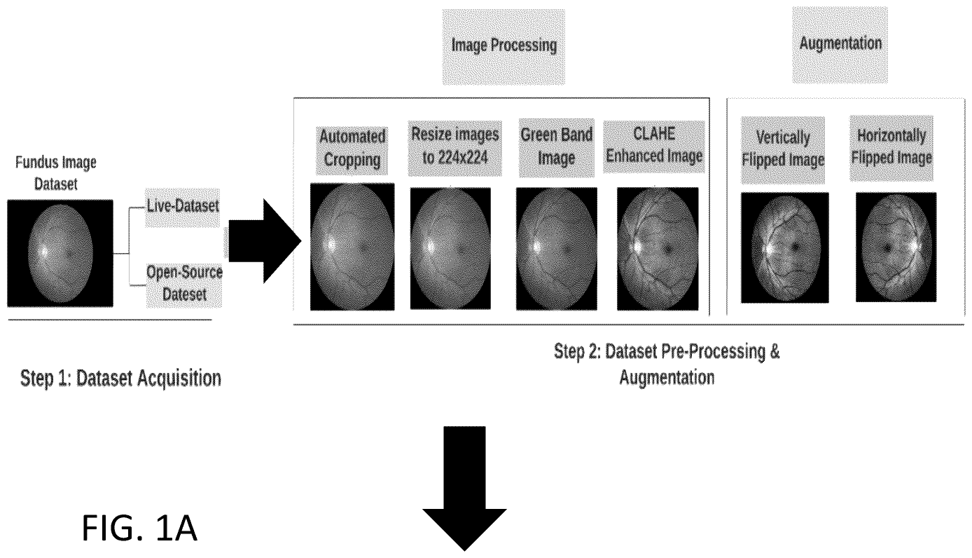

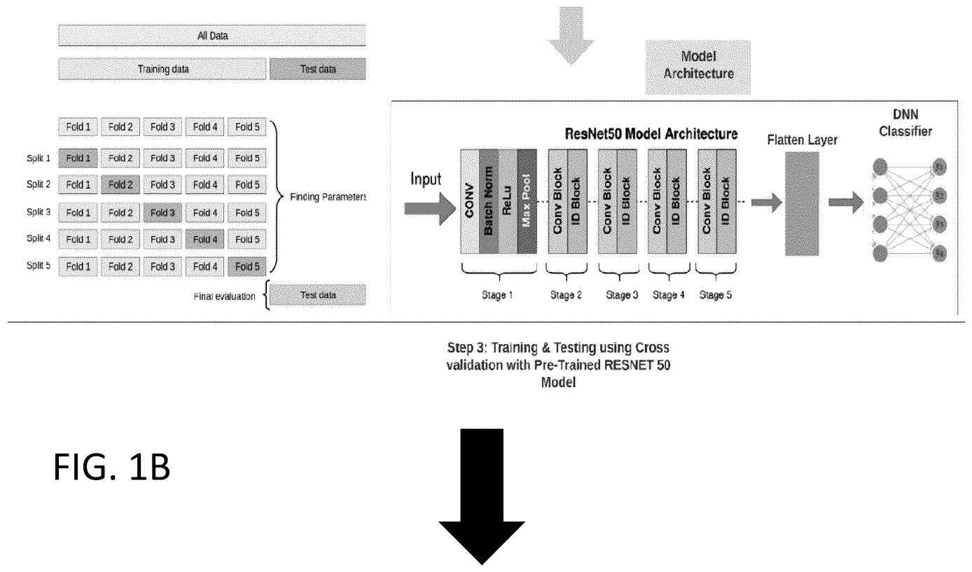

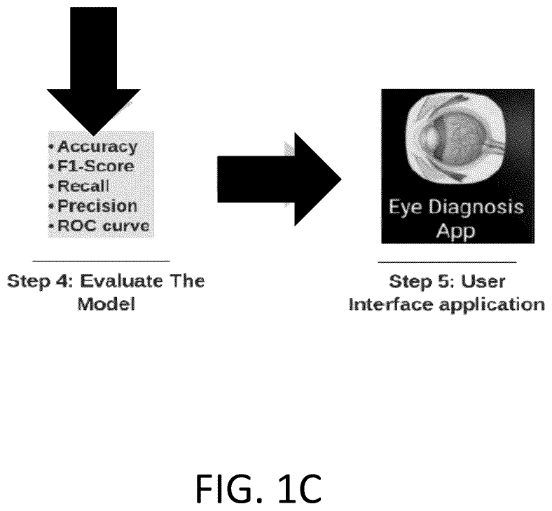

FIGS. 1A, 1B, and 1C further describe the five stages of the processes. As shown in FIG. 1A, dataset acquisition occurs in step 1. In embodiments, the data acquisition may be from a live dataset (e.g., images taken from human beings inside the hospital) or from an open-source dataset. As shown in FIG. 1A, dataset preprocessing and augmentation conducted in step 2. In embodiments, the preprocessing of step 2 includes automated cropping, image resizing, green band image, and CLAHE enhanced image. In embodiments, the augmentation portion of step 2 includes vertically flipped image and horizontally flipped image. In embodiments, a fundus image may come with a black rectangular background. In embodiments, cropping removes the unnecessary black areas so only the circular retina remains.

Accordingly, the cropping makes the image cleaner, removes noise and allows the ocular analysis system to focus on a retina image. In embodiments, all images are resized to 224×224 so they can fit the ResNet-50 input size, reduce computational load, and become uniform for the model. In embodiments, the fund images are RGB. In embodiments, a green band image (green channel extraction) occurs since the ocular analysis system uses a green channel to highlight blood vessels, lesions, and exudates. In embodiments, the ocular analysis system learns the disease feature best using the green channel.

In embodiments, CLAHE (Contrast Limited Adaptive Histogram Equalization) purpose is to increase contrast, enhance visibility of vessels, macula, optic disc, makes small lesions more obvious which is in DR and AMD detection. In embodiments, augmentation (horizontal and vertical flip) is used as AMD and glaucoma events have fewer images. In embodiments, flopping is used to creates new variations from the same image, helps the model generalize, and prevents overfitting.

FIG. 1B describes the training and testing cross validation a pre-trained model. In embodiments, the pre-trained model uses transfer lending in which the pre-trained model extracts features In embodiments, the stratified K-Fold cross validation ensures all classes (Normal, DR, AMD, Glaucoma) appear in each fold to ensure no class imbalance in training/testing and more reliable accuracy. In embodiments, each fold trains on 90% and tests on 10% of images. For example, the testing set used may be 677 images (10%). In embodiments, the model is evaluated using accuracy, ROC Curve (AUC), and confusion matrix.

FIG. 1C describes the fourth and fifth steps. In embodiments, the fourth step includes evaluating the model and the fifth step includes a user interface application which uses the model to analyze inputted eye data.

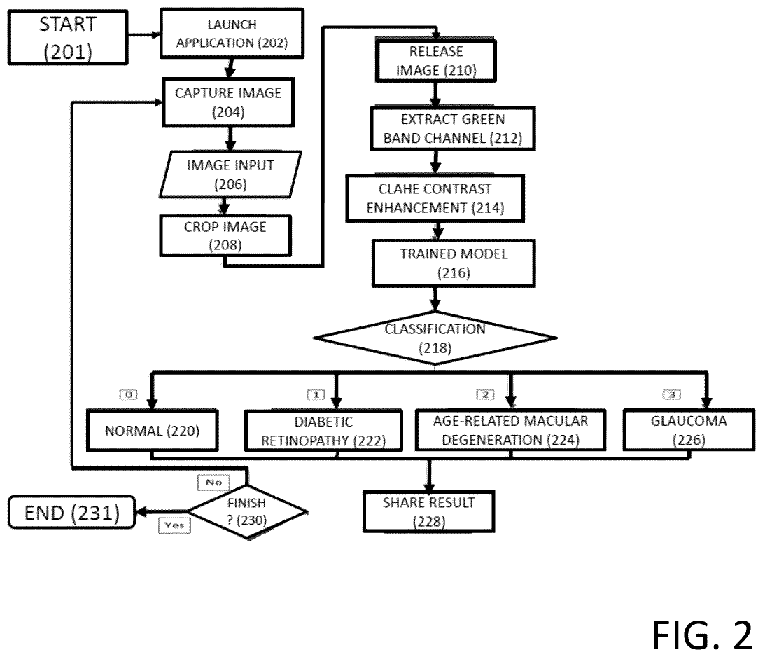

FIG. 2 describes a flow process for analyzing eye data information. In embodiments, the flow diagram represents the process of retinal image analysis and diagnosis and describes the sequential steps involved in capturing, processing, and classifying retinal images to identify potential eye diseases. This perspective focuses on the workflow followed by healthcare professionals or individuals involved in retinal imaging and diagnosis. At step 1, patient examination and image capture occurs. In embodiments, a healthcare professional prepares a 3D-printed gadget (smartphone+20D lens), and also a patient, in a darkened room or proper lighting. In embodiments, the fundus is visualized using a smartphone camera as the imaging device. In embodiments, a two-dimensional ophthalmology lens mounted on the gadget. In embodiments, a smartphone flashlight focuses through a small tube to illuminate the retina.

In embodiments, the retinal image is captured and is then sent to the ocular analysis system. In embodiments, at step 2, a user can use to capture an image (via the smartphone camera) or can be uploaded. For example, an image can be uploaded from a drive or other type of electronic source. In embodiments, capturing a new image includes using the electronic application to record a video of the retina, play the video, pause at the best frame, and then take a screenshot.

In embodiments, at step 3, circular cropping of the image occurs. In embodiments, once a good frame or image is ready, the user opens the image in the app's cropping screen and uses a circular cropping tool to center the optic disc and macula region, remove eyelids, eyelashes, extra black background, and confirms to get the cropped fundus image. In embodiments, the cropping makes the retina the main region of interest (ROI), reduces noise (eyelids, borders, reflections). ensures the model sees only the relevant anatomical area.

In embodiments, at step 4, automatic preprocessing is conducted. After cropping, the electronic application sends the image to the ocular analysis system, where preprocessing runs automatically. In embodiments, the ocular analysis system resizing image to 224×224 pixels. In embodiments, green channel extraction occurs in which the image is converted to RGB channels. In embodiments, the green channel highlights blood vessels, macula, lesions (e.g., exudates, hemorrhages, etc.). In embodiments, CLAHE (Contrast Limited Adaptive Histogram Equalization) on the green channel enhances contrast between vessels vs background, diseased vs healthy areas, and makes features more visible to the model. In embodiments, image processing/preprocessing makes all incoming images standardized, meaning the same size, similar brightness/contrast, and enhanced disease features which ensures the incoming test image is processed in the same way as the training images.

At step 5, the image is passed into the trained model. In embodiments, convolutional layers of ResNet-50 act like a “feature detector.” In embodiments, the layers of ResNet-50 learn patterns for blood vessel shape, spots, hemorrhages, exudates, optic disc cupping (glaucoma), and macular changes (AMD, DR). In embodiments, classification layers consist of flattening a layer, fully connected layers, and then generates a softmax output. In embodiments, the output is a probability for each class, which includes normal, diabetic retinopathy, AMD, and glaucoma. In embodiments, the class with the highest probability is chosen as the diagnosis. In embodiments, this block in the flow is shown as DL model/ResNet-50 classifier.

In embodiments, at step 6, the result is returned to the electronic application. Once the ocular detection system finishes classification, the ocular detection system sends the result back to the electronic application. In embodiments, the electronic application displays a diagnosis lable (e.g., “Glaucoma detected” or “Normal”). At step 7, a medical professional (or similar individual) may use the outcome of the electronic application as a primary analysis before any analysis conducted by the medical professional themselves.

Accordingly, the flow diagram in FIG. 2 illustrates the complete retinal image analysis workflow from the perspective of the healthcare professional. First, a fundus image is captured using a smartphone integrated with a 20D ophthalmic lens and a custom 3D-printed gadget that stabilizes the lens and focuses the phone flashlight on the retina. The user may either upload a previously captured fundus image or acquire a new one through the Eye Diagnosis application. The selected frame is then manually circularly cropped to isolate the region of interest and remove irrelevant background structures. The cropped image is transmitted to a Flask server, where it undergoes automated preprocessing, including resizing to 224×224 pixels, green-channel extraction, and CLAHE-based contrast enhancement to highlight retinal structures. The preprocessed image is subsequently input to a pre-trained ResNet-50 deep learning model, which performs multi-class classification into Normal, Diabetic Retinopathy, Age-Related Macular Degeneration, or Glaucoma. Finally, the predicted diagnosis is sent back to the mobile application and displayed to the user, thereby completing the end-to-end workflow from image acquisition to AI-assisted ocular disease screening.

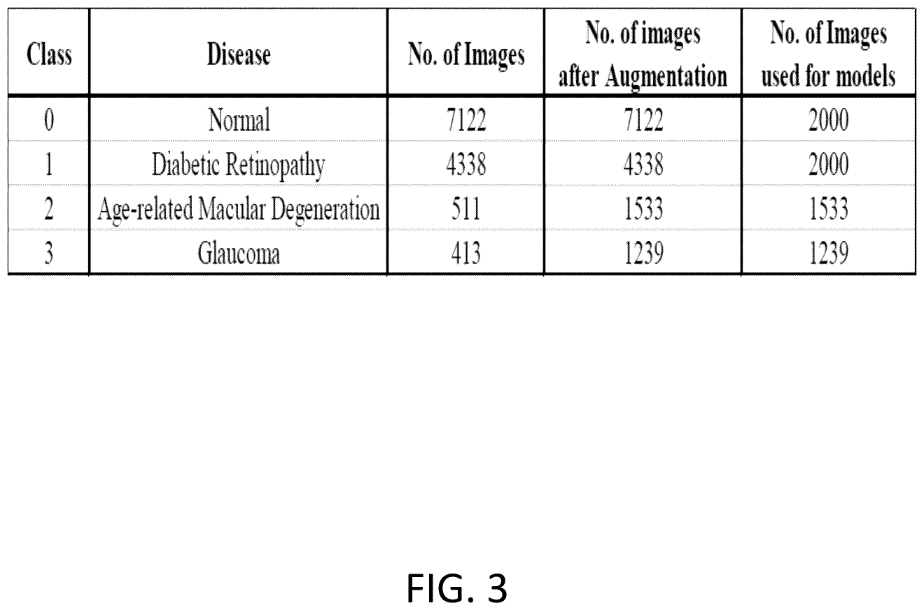

FIG. 3 describes a table that provides example numbers of images that can be used for the four classes which include Normal images, Diabetic Retinopathy images, Age-related Macular Degeneration images, and Glaucoma images. For example, a total of 12,384 images may be curated from these datasets that belong to the studied four classes as shown in FIG. 3. Data augmentation can be performed to increase the number of images for classes 2 and 3. In addition, the number of images for classes 0 and 1 can be reduced to 2,000 to be similar to the number of images in the other classes. This is to eliminate bias from the model classification, as the training data for one specific class increases, its more probable for the deep learning models to predict newly introduced data as that class.

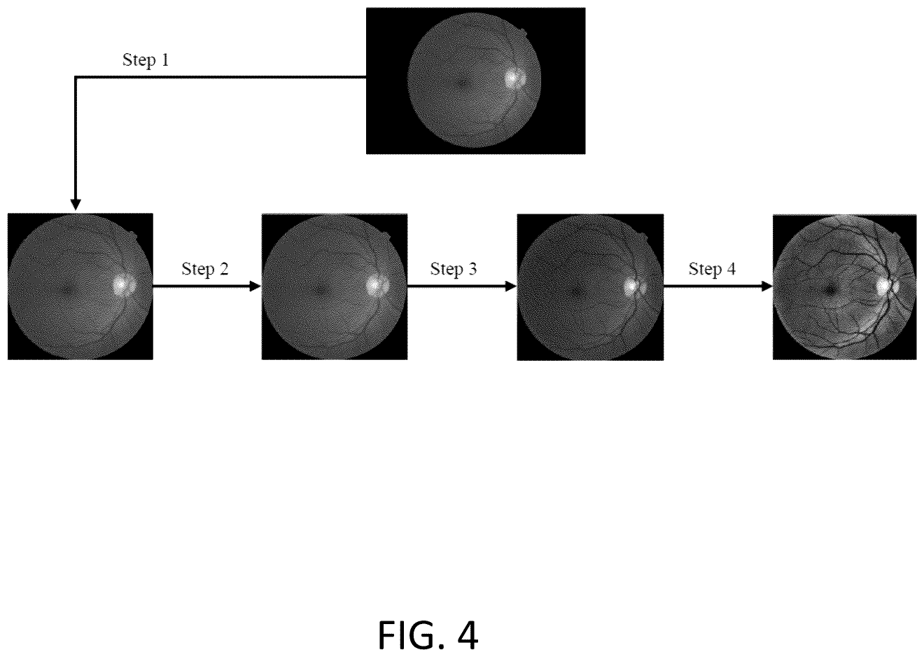

FIG. 4 describes image preprocessing which occurs due to the use of multiple datasets each with a different configuration and image characteristics. Even though all collected images are raw (unprocessed) fundus images, differences can occur due to the system that is used to capture the image, the lighting settings of the image environment, and the standardized size of the image at the medical center where the image is captured. In embodiments, image preprocessing technique is applied to all the images before they are introduced to the deep learning models is essential. Thus, the resulting images will all have the same configurations and characteristics.

Fundus images when captured using a fundus camera which has a rectangular configuration where the fundus image is centered and surrounded by a black background. This creates an issue when resizing the images, so the first step in the image preprocessing is to crop the black background out of the image, as shown in step 1, leaving only the fundus image with a square black background. Thus, steps 2 to 4 describe the update to an fundus image from an unprocessed image through each of the preprocessing steps until being ready for introduction for further processing by the ocular analysis system.

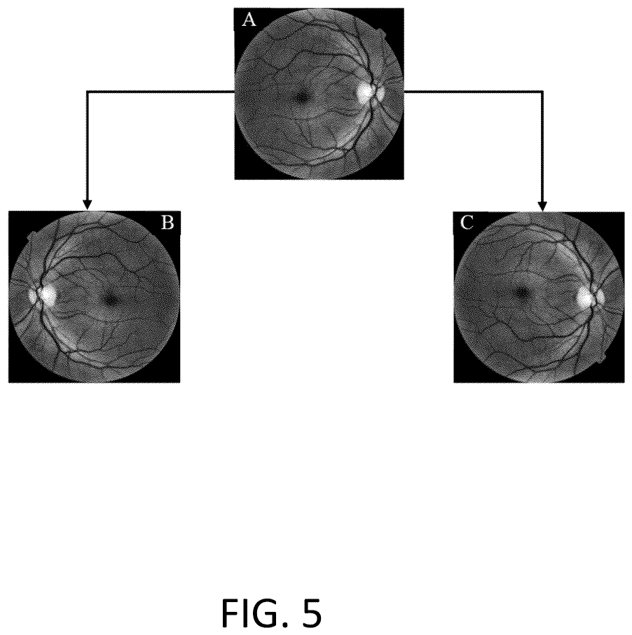

FIG. 5 describes further processing of ocular images which includes further highlighting the contrast between the different structures of the retinal image. In embodiments, as discussed in FIG. 4, this is achieved by first extracting the green channel from the RGB channel. Then, CLAHE is applied to the green channel which results in the last image seen in FIG. 4. In FIG. 5, with regards to data augmentation, each image, after image preprocessing is applied and produces two new images. As shown in FIG. 5, one is a horizontally flipped version of the original image, while the second is a vertically flipped version of the original image as a way of augmenting the data and to increase the number of images in the dataset to increase the training set for the model to increase the accuracy of the model)

Data augmentation was necessary for this project as the number of images collected for the AMD and Glaucoma diseases is lower than Normal and DR images, which if introduced to the model as is, will create a biased learning experience for the model. However, only two new images are created for each image as redundancy in the dataset is to be avoided. In embodiments, the number of online datasets for AMD and Glaucoma is lower than the number of online datasets for Diabetic Retinopathy).

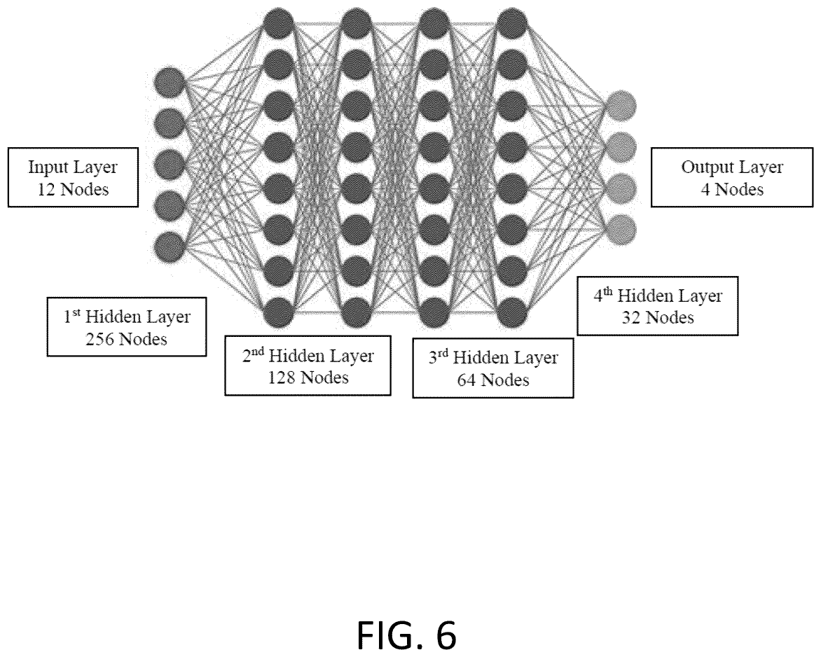

FIG. 6 describes one of two deep learning (DL) models used to train and test the collected and processed images. In embodiments, as shown in FIG. 6, the first model is a developed Deep Neural Network (DNN) model. Twelve features were extracted from the datasets which were then introduced into the hidden layers. The twelve features are the following: Mean, Standard Deviation, Skewness, Kurtosis, Contrast, FFT Mean, Entropy of the Histogram, Energy of the Histogram, Dissimilarity of the GLCM image, Homogeneity of the GLCM image, and Energy of the GLCM image. In embodiments, the model is composed of a 12-dimensional input layer, 4 fully connected hidden layers with the activation function Rectified Linear Unit (ReLU), and an output layer with 4 neurons and a softmax activation function to allow classification into the four diagnosis classes. In embodiments, the model was trained for 2000 epochs and with a batch size of 32. StratifiedKFold cross-validation technique was implemented over 10 folds to ensure that the number of images of each class is equal in each fold and to prevent the bias of Random Splitting.

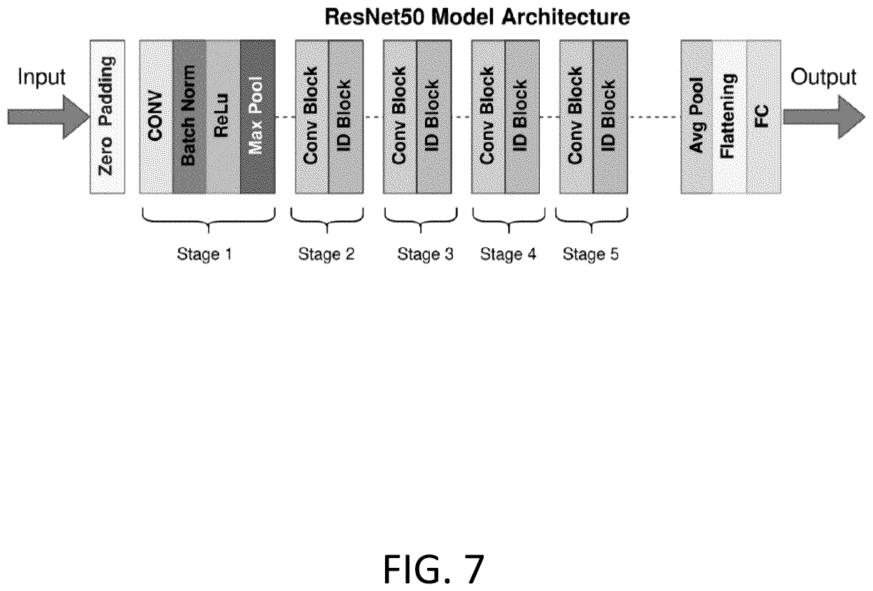

FIG. 7 describes the second model that is used as part of the ocular analysis system. In embodiments, the second utilized model is the ResNet-50 pre-trained Convolutional Neural Network (CNN) model. Feature Extraction: The ResNet50 model serves as the feature extractor in this architecture. It is a deep convolutional neural network pretrained on the ImageNet dataset. The purpose of this part is to extract high-level features from input images. ResNet50 comprises several convolutional layers organized into blocks, including convolutional layers, max-pooling layers, and identity blocks. These layers are responsible for learning hierarchical representations of the input images, capturing patterns and structures at various levels of abstraction.

In embodiments, following the ResNet50 base, additional layers are added for classification. These layers are responsible for transforming the extracted features into predictions for the target classes. In this specific architecture, a Flatten layer is used to flatten the output feature maps into a 1D array, followed by a Dense layer with ReLU activation for feature transformation. Finally, a Dense layer with softmax activation is added for multi-class classification, outputting class probabilities. The ResNet50 model is utilized as a feature extractor in a transfer learning setup. Here is how the ResNet50 model is used and the purpose of each layer in the model.

In embodiments, the ResNet50 model is loaded with pre-trained weights from the ImageNet dataset. By including includetop=False, only the convolutional base of the model is loaded, excluding the fully connected layers. All layers of the pre-trained ResNet50 model are frozen (layer.trainable=False), meaning their weights are not updated during training. This is done to prevent the pre-trained weights from being modified while training the new classification layers. In embodiments, a new Sequential model is created, where the ResNet50 base model serves as the first layer. Following the ResNet50 base, a Flatten layer is added to flatten the output feature maps into a 1D array. Then, a Dense layer with ReLU activation is added for feature transformation, and finally, a Dense layer with softmax activation is added for classification.

In embodiments, the model is compiled with appropriate loss function, optimizer, and evaluation metric for the classification task. In embodiments, the data is split into training and testing sets using stratified K-fold cross-validation to ensure balanced class distribution in each fold. In embodiments, the model is trained using the training data. During training, ModelCheckpoint callback is used to save the best model weights based on validation accuracy.

After each fold of training, the model is evaluated on the corresponding test set. A classification report is generated to assess & evaluate the model performance in terms of precision, recall, accuracy, and F1-score for each class. Once the cross-validation is complete, the final trained model is saved for future use. Accordingly, the ResNet-50 model serves as a feature extractor. The convolutional layers of ResNet50 are utilized to extract meaningful features from input images, and then layers are added for classification on top of those extracted features. In embodiments, the power of transfer learning is leveraged, where the pre-trained ResNet50 model has already learned rich hierarchical representations from a large dataset like ImageNet, and fine-tuned for our specific classification task.

The testing set that was used to evaluate both models had 10% of the total images (e.g., 677 images of 6772). Furthermore, the Area Under the Receiver Operating Characteristic Curve (AUC-ROC) was also used to evaluate the models. This is used to display the statistical probabilities of the model to predict the input image correctly for each class. It is generally higher than the accuracy of the models' predictions. The average accuracy and ROC of both models across all folds were compared to choose the best fit for the final system.

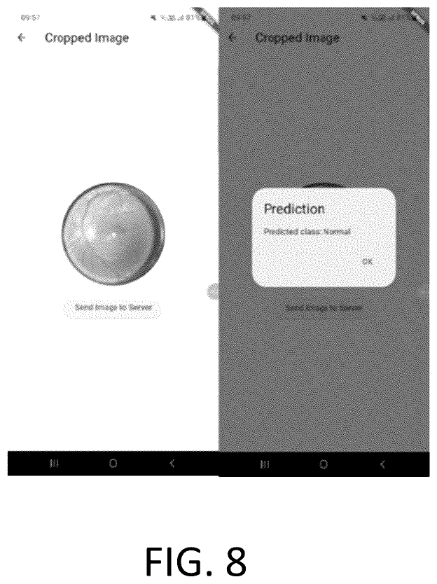

FIG. 8 describes an example screen shot of an ocular disease classification application which is a user-friendly mobile application designed to help detect and classify various ocular diseases like Glaucoma, Age-related Macular Degeneration (AMD), and Diabetic Retinopathy. In embodiments, users can use the electronic application easily capture or upload images of the retinas, crop them to focus on the important areas, and submit them for analysis.

In embodiments, the ocular disease classification application interface makes it easy to navigate through each step—from capturing and uploading images to cropping and receiving results. By using deep learning and maintaining a user-friendly design, the ocular disease classification application (using the ocular analysis system) empowers users to take charge of their eye health. This enables early detection, ultimately contributing to better health outcomes.

In embodiments, the DNN model achieved an average accuracy of 0.915 or 91.5% and a standard deviation of 0.0077 when tested on the Testing set on each of the ten folds. The predictions of the model were compared with the ground truths of the testing set and the accuracy was calculated. The ResNet-50 CNN model achieved an average accuracy of 0.966 or 96.6% and a standard deviation of 0.01 when tested on the Testing set on each of the ten folds. The predictions of the model were compared with the ground truths of the testing set and the accuracy was calculated.

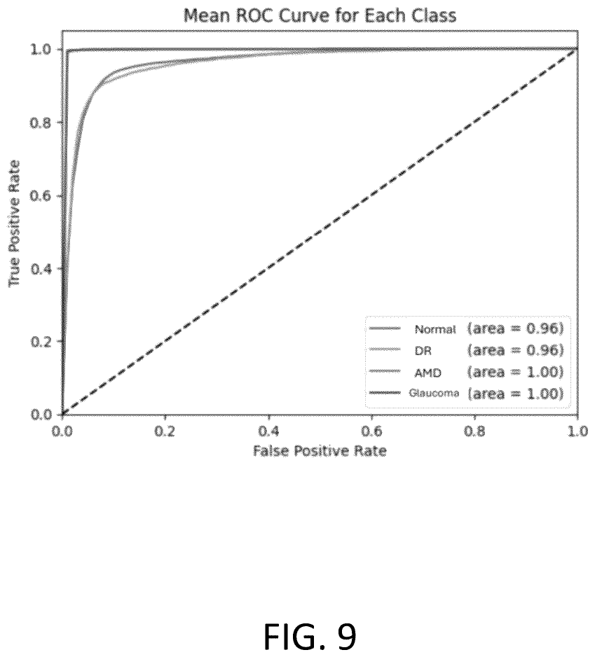

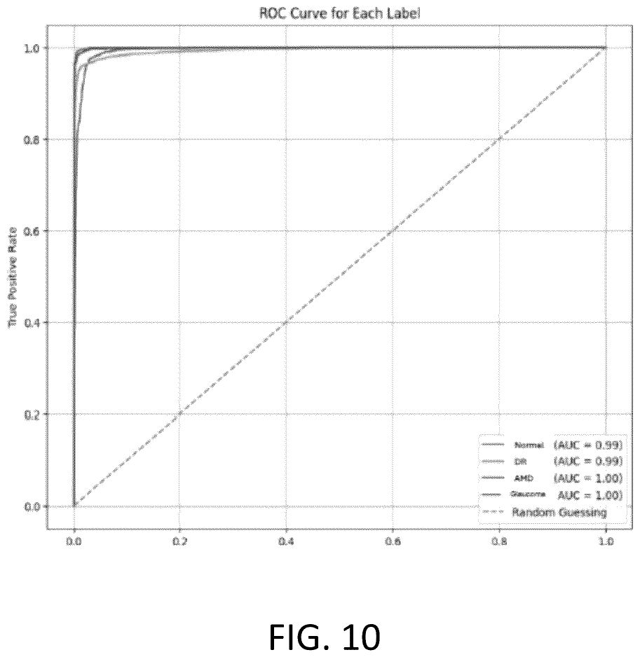

FIGS. 9 and 10 shows the average AUC-ROC for both models with FIG. 9 for the DNN model and FIG. 10 for the ResNet-50 model.

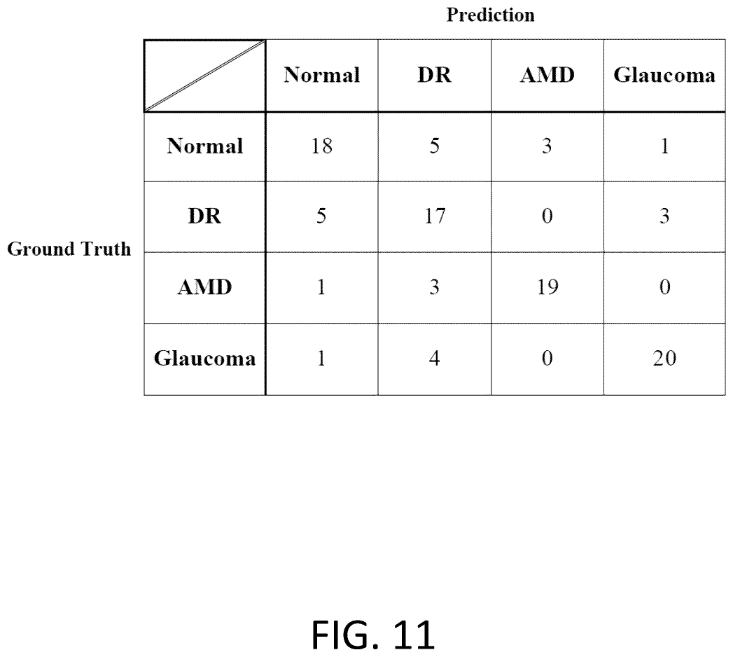

FIG. 11 describes an example manual testing dataset. As shown in FIG. 11, a manual testing dataset is established that included 25 images for each class. In embodiments, the manual testing dataset shows how the AI models in the ocular detection system performs on new, unseen images. In embodiments, the manual testing dataset ensures that a model does not overfit. In embodiments, the testing on images may not be used in training. In embodiments, the manual testing dataset includes from different sources, such as unused dataset images, external dataset, and/or live data from a user device. In embodiments, the manual testing dataset helps to compare the results to results from other types of systems.

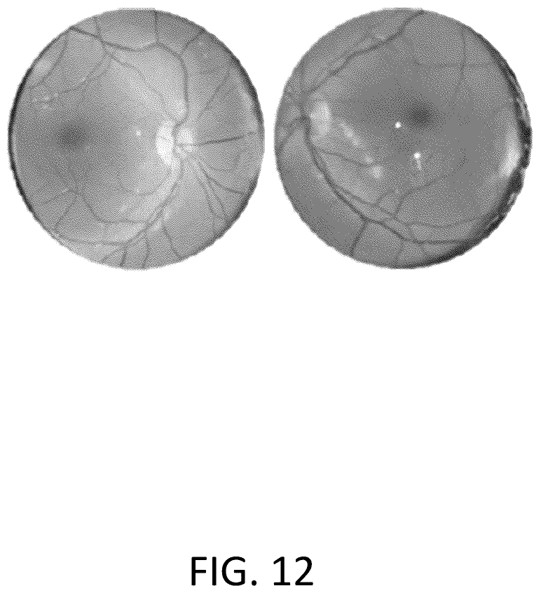

FIG. 12 describes example fundus images captured by the proposed system. Both images are examples of Normal diagnosis images and are free of any diseases. In embodiments, one of the main difference between the proposed system and the previous systems is that the previous systems implemented a binary classification approach. On the other hand, the proposed system implemented a multi-classification approach which offers a wider diagnostic capability that signifies a progress in the intricacy and range of ocular disease identification while maintaining an accuracy of 74%.

When comparing the cost of the developed system and the conventional systems for capturing fundus images, the developed system performs much better than conventional fundus cameras or slit lamps as it has a considerably less cost of manufacturing. The total cost of the system is less than 550 USD. In comparison, the cost of a fundus camera is at least 2,000 USD. Furthermore, the ocular analysis system is lightweight, portable, has AI capabilities, and targets specific eye diseases.

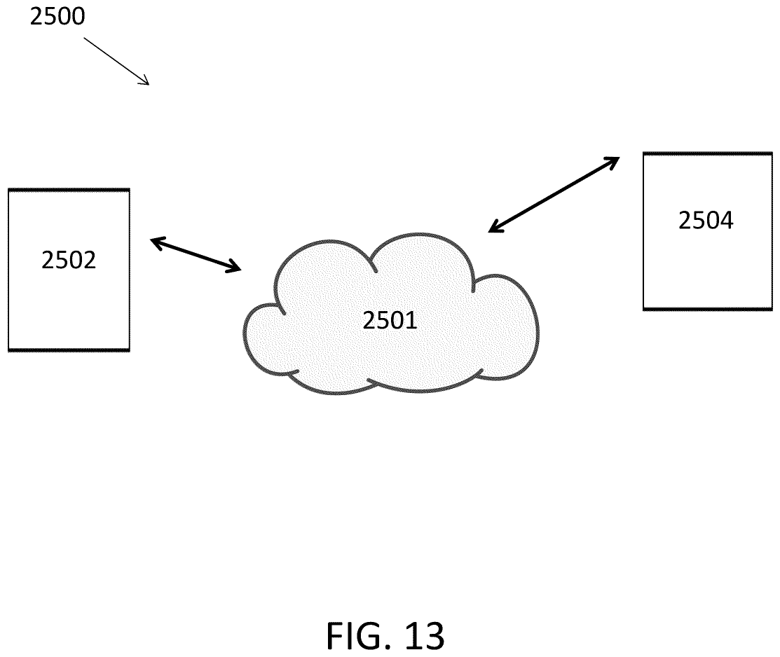

FIG. 13 is a diagram of example environment 2500 in which systems, devices, and/or methods described herein may be implemented. FIG. 5 shows network 2501, user device 2502, and system 2504.

Network 2501 may include a local area network (LAN), wide area network (WAN), a metropolitan network (MAN), a telephone network (e.g., the Public Switched Telephone Network (PSTN)), a Wireless Local Area Networking (WLAN), a WiFi, a hotspot, a Light fidelity (LiFi), a Worldwide Interoperability for Microware Access (WiMax), an ad hoc network, an intranet, the Internet, a satellite network, a GPS network, a fiber optic-based network, and/or combination of these or other types of networks. Additionally, or alternatively, network 500 may include a cellular network, a public land mobile network (PLMN), a second generation (2G) network, a third generation (3G) network, a fourth generation (4G) network, a fifth generation (5G) network, and/or another network.

In embodiments, network 2501 may allow for devices describe any of the described figures to electronically communicate (e.g., using emails, electronic signals, URL links, web links, electronic bits, fiber optic signals, wireless signals, wired signals, etc.) with each other so as to send and receive various types of electronic communications.

User device 2502 may include any computation or communications device that is capable of communicating with a network (e.g., network 2501). For example, user device 2502 may include a radiotelephone, a personal communications system (PCS) terminal (e.g., that may combine a cellular radiotelephone with data processing and data communications capabilities), a personal digital assistant (PDA) (e.g., that can include a radiotelephone, a pager, Internet/intranet access, etc.), a smart phone, a desktop computer, a laptop computer, a tablet computer, a camera, a personal gaming system, a television, a set top box, a digital video recorder (DVR), a digital audio recorder (DUR), a digital watch, a digital glass, or another type of computation or communications device.

User device 2502 may receive and/or display content. The content may include objects, data, images, audio, video, text, files, and/or links to files accessible via one or more networks. Content may include a media stream, which may refer to a stream of content that includes video content (e.g., a video stream), audio content (e.g., an audio stream), and/or textual content (e.g., a textual stream). In embodiments, an electronic application may use an electronic graphical user interface to display content and/or information via user device 2502. User device 2502 may have a touch screen and/or a keyboard that allows a user to electronically interact with an electronic application. In embodiments, a user may swipe, press, or touch user device 2502 in such a manner that one or more electronic actions will be initiated by user device 2502 via an electronic application. User device 2502 may receive electronic information from antenna 506 and generate and display graphs such as those described in the figures above.

User device 2502 may include a variety of applications, such as, for example, an ocular disease classification application, e-mail application, a telephone application, a camera application, a video application, a multi-media application, a music player application, a visual voice mail application, a contacts application, a data organizer application, a calendar application, an instant messaging application, a texting application, a web browsing application, a blogging application, and/or other types of applications (e.g., a word processing application, a spreadsheet application, etc.). In embodiments, user device 2502 may be used to generate images associated with various types of eye diseases.

System 2504 may be any computation or communications device that is capable of communicating with a network (e.g., network 2501) and user device 2502. In embodiments, system 2504 may be ocular analysis system and may include one or more devices that can conduct the numerous features of the ocular analysis system as described in FIGS. 1 to 13.

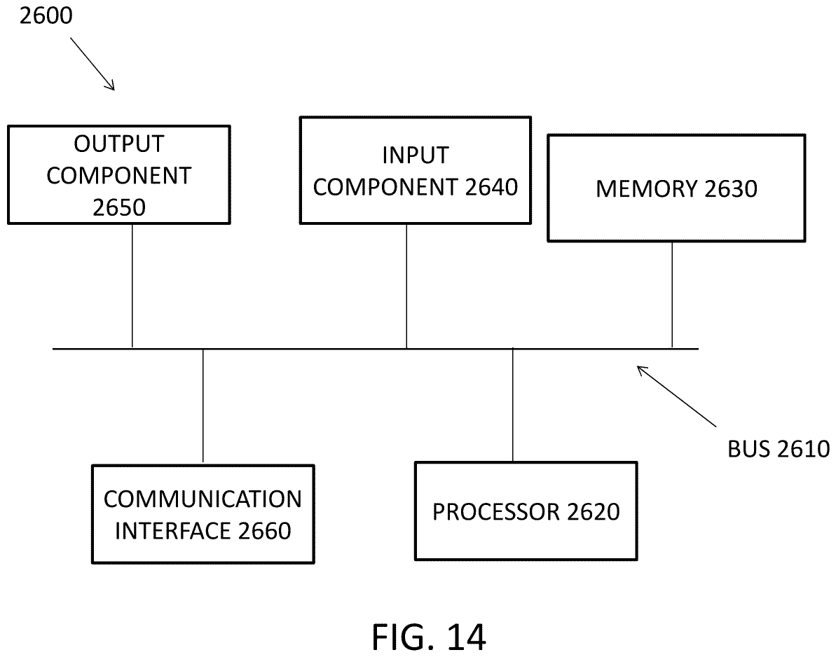

FIG. 14 is a diagram of example components of a device 2600. Device 2600 may correspond to user device 2502, or system 2504. Alternatively, or additionally, user device 2502 and system 2504 may include one or more devices 2600 and/or one or more components of device 2600.

As shown in FIG. 14, device 2600 may include a bus 2610, a processor 2620, a memory 2630, an input component 2640, an output component 2650, and a communications interface 2660. In other implementations, device 2600 may contain fewer components, additional components, different components, or differently arranged components than depicted in FIG. 14. Additionally, or alternatively, one or more components of device 2600 may perform one or more tasks described as being performed by one or more other components of device 2600.

Bus 2610 may include a path that permits communications among the components of device 2600. Processor 2620 may include one or more processors, microprocessors, or processing logic (e.g., a field programmable gate array (FPGA) or an application specific integrated circuit (ASIC)) that interprets and executes instructions. Memory 2630 may include any type of dynamic storage device that stores information and instructions, for execution by processor 2620, and/or any type of non-volatile storage device that stores information for use by processor 2620. Input component 2640 may include a mechanism that permits a user to input information to device 2600, such as a keyboard, a keypad, a button, a switch, voice command, etc. Output component 2650 may include a mechanism that outputs information to the user, such as a display, a speaker, one or more light emitting diodes (LEDs), etc.

Communications interface 2660 may include any transceiver-like mechanism that enables device 2600 to communicate with other devices and/or systems. For example, communications interface 2660 may include an Ethernet interface, an optical interface, a coaxial interface, a wireless interface, or the like.

In another implementation, communications interface 2660 may include, for example, a transmitter that may convert baseband signals from processor 2620 to radio frequency (RF) signals and/or a receiver that may convert RF signals to baseband signals. Alternatively, communications interface 660 may include a transceiver to perform functions of both a transmitter and a receiver of wireless communications (e.g., radio frequency, i2nfrared, visual optics, etc.), wired communications (e.g., conductive wire, twisted pair cable, coaxial cable, transmission line, fiber optic cable, waveguide, etc.), or a combination of wireless and wired communications.

Communications interface 2660 may connect to an antenna assembly (not shown in FIG. 26) for transmission and/or reception of the RF signals. The antenna assembly may include one or more antennas to transmit and/or receive RF signals over the air. The antenna assembly may, for example, receive RF signals from communications interface 2660 and transmit the RF signals over the air, and receive RF signals over the air and provide the RF signals to communications interface 2660. In one implementation, for example, communications interface 2660 may communicate with network 2501.

As will be described in detail below, device 2600 may perform certain operations. Device 600 may perform these operations in response to processor 2620 executing software instructions (e.g., computer program(s)) contained in a computer-readable medium, such as memory 2630, a secondary storage device (e.g., hard disk, CD-ROM, etc.), or other forms of RAM or ROM. A computer-readable medium may be defined as a non-transitory memory device. A memory device may include space within a single physical memory device or spread across multiple physical memory devices. The software instructions may be read into memory 2630 from another computer-readable medium or from another device. The software instructions contained in memory 2630 may cause processor 2620 to perform processes described herein. Alternatively, hardwired circuitry may be used in place of or in combination with software instructions to implement processes described herein. Thus, implementations described herein are not limited to any specific combination of hardware circuitry and software.

FIGS. 15A, 15B, and 15C are diagrams that describe a holding device. FIG. 15A describes a perspective view of the holding device, FIG. 15B describes a top view of the holding device, and FIG. 15C describes a side view of the holding device.

It will be apparent that example aspects, as described above, may be implemented in many different forms of software, firmware, and hardware in the implementations illustrated in the figures. The actual software code or specialized control hardware used to implement these aspects should not be construed as limiting. Thus, the operation and behavior of the aspects were described without reference to the specific software code—it being understood that software and control hardware could be designed to implement the aspects based on the description herein.

Even though particular combinations of features are recited in the claims and/or disclosed in the specification, these combinations are not intended to limit the disclosure of the possible implementations. In fact, many of these features may be combined in ways not specifically recited in the claims and/or disclosed in the specification. Although each dependent claim listed below may directly depend on only one other claim, the disclosure of the possible implementations includes each dependent claim in combination with every other claim in the claim set.

While various actions are described as selecting, displaying, transferring, sending, receiving, generating, notifying, and storing, it will be understood that these example actions are occurring within an electronic computing and/or electronic networking environment and may require one or more computing devices, as described in FIG. 13, to complete such actions.

No element, act, or instruction used in the present application should be construed as critical or essential unless explicitly described as such. Also, as used herein, the article “a” is intended to include one or more items and may be used interchangeably with “one or more.” Where only one item is intended, the term “one” or similar language is used. Further, the phrase “based on” is intended to mean “based, at least in part, on” unless explicitly stated otherwise.

In the preceding specification, various preferred embodiments have been described with reference to the accompanying drawings. It will, however, be evident that various modifications and changes may be made thereto, and additional embodiments may be implemented, without departing from the broader scope of the invention as set forth in the claims that follow. The specification and drawings are accordingly to be regarded in an illustrative rather than restrictive sense.

What is claimed is:

1. A method, comprising:
receiving, by an apparatus, electronic information about an eye;
cropping, by the apparatus, the electronic information;
analyzing, by the apparatus, the electronic information; and
determining, by the computing system, that the electronic information is associated with one of:
a healthy eye, or an unhealthy eye.

2. The method of claim 1, wherein the apparatus is configured to include a holding device, wherein the holding device holds a user device.

3. The method of claim 2, wherein the apparatus includes a cylindrical feature through which an image of an eye is captured.

4. The method of claim 1, wherein the analyzing the electronic information includes generating a green band image.

5. The method of claim 1, wherein the analyzing the electronic information includes generating horizontal and vertical images of the electronic information.

6. An apparatus, comprising:
a cylindrical section;
a slidable section, wherein a user device is configured to fit between the cylindrical section and the slidable section;
a vertical rod, wherein the vertical rod is used as a handle; and
a horizontal rod, wherein the horizontal rod is configured to move the slidable section.
